(19) United States
(12) Patent Application Publication
(10) Pub. No.: US 2025/0261882 A1
(43) Pub. Date: Aug. 21, 2025

(54) MEDICAL DEVICE FOR DETECTING AT LEAST ONE ANALYTE IN A BODY FLUID

(71) Applicant: Roche Diabetes Care, Inc., Indianapolis, IN (US)

(72) Inventor: Felix Bootz, Speyer (DE)

(21) Appl. No.: 19/203,861

(22) Filed: May 9, 2025

Related U.S. Application Data
(63) Continuation of application No. PCT/EP2023/081553, filed on Nov. 13, 2023.

(30) Foreign Application Priority Data
Nov. 14, 2022 (EP) 22 207 361.1

(51) Int. Cl. A61B 5/1473; A61B 5/00

(52) U.S. Cl. CPC A61B 5/1473; A61B 5/6833

(57) ABSTRACT

A medical device for detecting an analyte in a body fluid has a base body for reversibly attaching the medical device to a patient's body and has an insertable analyte sensor with first and second electrodes. An electronics unit is connected to the sensor. The first and second electrodes each have a first free end and a second end, the second ends being electrically connected to respective electric conductors connecting the respective electrodes to the electronics unit. The first electrode extending from the second end and the second electrode extending from the second end are at least partially in electric contact with the body tissue of a patient during use of the medical device. The electrical resistance of the first electric conductor and the electrical resistance of the second electric conductor is higher than the electrical resistance of the first electrode and the electrical resistance of the second electrode.

### RELATED APPLICATIONS

This application is a continuation of International Application No. PCT/EP2023/081553, filed Nov. 13, 2023, which claims priority to European Application No. 22 207 361.1, filed Nov. 14, 2022, the entire disclosures of both of which are hereby incorporated herein by reference.

### BACKGROUND

This disclosure relates to a medical device for detecting at least one analyte in a body fluid. The device has a base body configured for reversibly attaching the medical device to a patient's body; an insertable analyte sensor comprising an insertable portion for being at least partially inserted into body tissue of a user; and an electronics unit operably connected to the insertable analyte sensor. The insertable analyte sensor has a first electrode and a second electrode, both forming part of the insertable portion. The first electrode has a first free end and a second end, wherein the second end of the first electrode is electrically connected to a first electric conductor electrically connecting the first electrode of the insertable analyte sensor to the electronics unit. The second electrode has a first free end and a second end, wherein the second end of the second electrode is electrically connected to a second electric conductor electrically connecting the second electrode of the insertable analyte sensor to the electronics unit, and wherein the first electrode extending from the second end towards its first free end and the second electrode extending from the second end towards its first free end are configured to be, during use of the medical device, at least partially in electric contact with the body tissue of a user.

A hitherto known medical device for detecting at least one analyte in a body fluid is, during use, removably attached to a user's body, wherein the insertable analyte sensor is inserted into the body tissue of the user. Accordingly, the first electrode and the second electrode are in direct contact with the body tissue, and electrically connected to the electronics unit. Thus, failure of the electronics unit, such a short circuit, may result in a high electric current flowing into the user's body via the first electrode and the second electrode. Therefore, in order to increase the user's safety, known medical devices have redundant electronic components such that a failure of an electronic component does not result in a high electric current flowing into the user's body, but is compensated by the respective redundant component. However, the number of extra, redundant electric components increases the production costs and results in a larger size of the medical device.

### SUMMARY

Accordingly, this disclosure teaches a medical device for detecting at least one analyte in a body fluid which avoids at least one disadvantage of hitherto known medical devices. In particular, this disclosure teaches a medical device for detecting at least one analyte in a body fluid that mitigates risk of a high current flowing into the body tissue of a user while allowing lower production costs.

According to a first aspect, a medical device for detecting at least one analyte in a body fluid, is provided, the medical device comprising a base body that is configured for reversibly attaching the medical device to a patient's body; an insertable analyte sensor comprising an insertable portion that is configured for being at least partially inserted into body tissue of a user; and an electronics unit that is operably connected to the insertable analyte sensor, wherein the insertable analyte sensor comprises a first electrode and a second electrode, both, forming part of the insertable portion, wherein the first electrode comprises a first free end and a second end, wherein the second end of the first electrode is electrically connected to a first electric conductor electrically connecting the first electrode of the insertable analyte sensor to the electronics unit, wherein the second electrode comprises a first free end and a second end, wherein the second end of the second electrode is electrically connected to a second electric conductor electrically connecting the second electrode of the insertable analyte sensor to the electronics unit, and wherein the first electrode extending from the second end towards its first free end and the second electrode extending from the second end towards its first free end are configured to be, during use of the medical device, at least partially in electric contact with the body tissue of a user, wherein the electrical resistance of the first electric conductor and the electrical resistance of the second electric conductor is higher than the electrical resistance of the first electrode and the electrical resistance of the second electrode.

The medical device for detecting at least one analyte in a body fluid can be removably attached to a user's body. For instance, the base body can be adhered to a user's skin. For example, the base body can comprise an adhesive layer or can comprise a patch comprising an adhesive that is configured for removably adhering the medical device to a user's body.

When being attached to a user's body, the insertable portion of the insertable analyte sensor is inserted into the body tissue of the user, wherein the first electrode and the second electrode are in electric contact with the body tissue and in electric contact with the electronics unit via the first conductor and the second conductor, respectively. Presence of an analyte in the body tissue will affect an electric current flowing between the first electrode and the second electrode. The change in electric current can be evaluated by the electronics unit so to allow a detection of the respective analyte. By way of example, only, the electronics unit can be a printed circuit board.

The analyte can be one or more analytes, preferably metabolites such as glucose, lactate, triglycerides, cholesterol or analytes in bodily fluids, such as, for example, blood or interstitial fluid or other bodily fluids. For instance, the insertable analyte sensor can be an electrochemical sensor that is configured for detecting at least one analyte, wherein the first electrode is configured as a working electrode and is used as transducer, and the second electrode is configured as a counter electrode.

The medical device can comprise a power supply that is configured for providing electric power to the electronics unit. The power supply can be a battery or a rechargeable battery that is configured for providing electric power to the electronics unit. By way of example, only, the battery can be configured to provide an electric power of 3.4 V.

In order to mitigate a risk of an uncontrolled electric current flowing into the user's body in case of a failure of the electronics unit or a component thereof, the electrical resistance of the first conductor and the electrical resistance of the second conductor is higher than the electrical resistance of the first electrode and the electrical resistance of the second electrode. During use, an electric current is flowing between the first electrode and the second electrode, the first electric conductor and the second electric conductor together with the first electrode and the second electrode form a potentiometer. The higher electrical resistance of the first conductor and the second conductor limits the electric current flowing through the first conductor and the second conductor, so that the electric current that is flowing through the first electrode and the second electrode is limited, too. Consequently, there is no need for redundant electric components that would limit the electric current flowing through the first electrode and the second electrode in case of a failure of the electronics unit or parts thereof. Without the need of redundant electric components, the overall number of electronic components of the electronics unit can be reduced, such that the medical device can be of smaller size and with lower production costs.

According to an embodiment, the sum of the electrical resistance of first electric conductor and the electrical resistance of the second electric conductor is higher than the sum of the electrical resistance of the first electrode and the electrical resistance of the second electrode. For instance, the sum of the electrical resistance of the first electrode and the electrical resistance of the second electrode can be 9 kOhm, while the sum of the electrical resistance of the first electrical conductor and the electrical resistance of the second electrical conductor can be 90 kOhm.

The electrical resistance of the first electrode and the electrical resistance of the second electrode can be equally high, and/or that the electrical resistance of the first electric conductor and the electrical resistance of the second electric conductor can be equally high. For the ease of its production, and for obtaining a symmetric potentiometer, it is beneficial that the electrical resistance of the first electrode is equal to the electrical resistance of the second electrode. Likewise, the electrical resistance of the first conductor is equal to the electrical resistance of the second conductor. In the meaning of this disclosure, equal means identical measurement values that, however, may deviate by a common measuring inaccuracy and production inaccuracy.

In an embodiment, the electrical resistance of the first electric conductor and the electrical resistance of the second electric conductor is at least 2-times, at least 4-times, at least 5-times or at least 10-times higher than the electrical resistance of the first electrode and the electrical resistance of the second electrode. For instance, the electrical resistance of the first electrode and electrical resistance of the second electrode each can be 9 kOhm, while the electrical resistance of the first electric conductor and the electrical resistance of the second electric conductor each can be 90 kOhm.

In case of a failure of the electronics unit or parts thereof, an electric current that is uncontrolled flowing via the first electrode and the second electrode into body tissue, may harm the body tissue if the current exceeds 50 uA. Therefore, the electrical resistance of the first electric conductor and the electrical resistance of the second electric conductor, that during use limit a maximum electric current flowing through the first electrode and the second electrode, are selected so that the voltage of the power supply divided by the sum of the electrical resistance of the first electrical conductor and the electrical resistance of the second electric conductor is smaller than or equal to 50 μA. By knowing the voltage of the power supply, e.g., the voltage of a battery, the electrical resistance of the first electric conductor and the electrical resistance of the second electric conductor can be determined so to limit the maximum electric current flowing through the first electrode and the second electrode. The sum of the electrical resistance of the first electric conductor and the electrical resistance of the second electric conductor can be equal to the voltage of the power supply divided by the maximum allowed electric current, here: 50 μA.

In an embodiment, the first electrode from its second towards its first free end and the second electrode from its second end towards its first free end are, over its respective length, configured to be during use of the medical device in electric contact with the body tissue. When being inserted, the first electrode and the second electrode forming at least part of the insertable portion, are in electric contact with the body tissue. At least one side of the respective first and second electrode, there is no electric isolation shielding the first and second electrode against the body tissue. On the other hand, the first electric conductor and the second electric conductor can be, over its respective length, configured to be during use of the medical device electrically isolated against the body tissue. Since, during use of the medical device, the first electric conductor and the second electric conductor limit the maximum electric current flowing through the first electrode and the second electrode, the first electric conductor and the second electric conductor should not be in direct electric contact with the body tissue.

According to an embodiment, the first electric conductor comprises a first cable cross-sectional area, the second electric conductor comprises a second cable cross-sectional area, the first electrode comprises a third cable cross-sectional area, and the second electrode comprises a fourth cable cross-sectional area, wherein the first cable cross-sectional area and the second cable cross-sectional area are smaller than the third cable cross-sectional area and the fourth cable cross-sectional area. The electrical resistance of the respective electric conductor or electrode is, inter alia, dependent on the cross-sectional area, wherein a smaller cross-sectional area provides a higher electrical resistance than a larger cross-sectional area of the same material. For instance, the cross-sectional area of the first electrode and the second electrode can be reduced by laser ablation.

The first cable cross-sectional area can be smaller than the third cable cross-sectional area, and the second cable cross-sectional area can be smaller than the fourth cable cross-sectional area. For instance, the first cable cross-sectional area and the second cable cross-sectional area are of equal size, and/or the third cable cross-sectional area and the fourth cable cross-sectional area are of equal size.

The first cable cross-sectional area and the second cable cross-sectional area can be at least 2-times, at least 4-times, at least 5-times, or at least 10-times smaller than the third cable cross-sectional area and the fourth cable cross-sectional area.

According to an embodiment, the base body comprises a bushing, wherein the electronics unit is arranged on a first side of the bushing and the insertable portion of the insertable analyte sensor is arranged on a second side of the bushing opposite to the first side, wherein the second end of the first electrode electrically connected to the first electric conductor, and the second end of the second electrode electrically connected to the second electric conductor are both located within the bushing or at the first side of the bushing comprising the electronics unit or at the second side of the bushing comprising the insertable portion of the insertable analyte sensor. The bushing has a first side and second side, and provides a channel for the first and second electric conductors and/or the first and second electrode which are electrically connected to the electronics unit. For instance, when located on the first side or within the bushing, a contact point between the first electrode and the first electric conductor as well as a contact point between the second electrode and the second electric conductor, i.e., the second end of the first electrode and/or the second end of the second electrode, is protected by the base body or the bushing. According to an embodiment, the bushing can be a through hole or duct in the base body allowing the first and second electric conductors and/or the first and second electrode to pass into and out of the base body. Like the base body, the bushing can be made from an electrical isolating material.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows a schematic view of a medical device for detecting at least one analyte in a body fluid according to an embodiment;

FIG. 2 shows a schematic view of a medical device for detecting at least one analyte in a body fluid according to another embodiment; and

FIG. 3 shows a schematic view of a medical device for detecting at least one analyte in a body fluid according to yet another embodiment.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

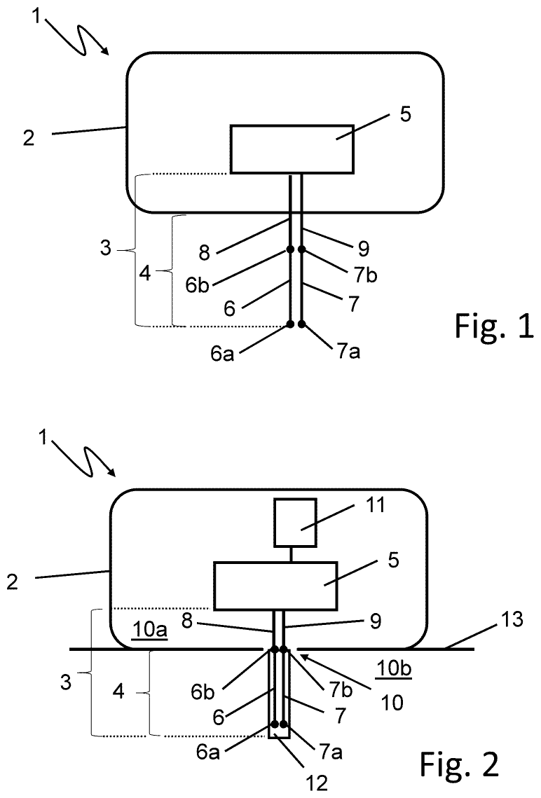

FIG. 1 shows a schematic view of a medical device 1. The medical device 1 is configured for detecting at least on analyte in a body fluid. The medical device 1 comprises a base body 2 that is configured for being removably attached to a patient's body. The base body 2 houses an electronics unit (also referred to herein as “electronics”) 5 that is operably connected to an insertable analyte sensor 3.

The insertable analyte sensor 3 comprises an insertable portion 4 that is configured for being at least partially inserted into body tissue of a user. The insertable portion 4 projects from the base body 2 so that it can be inserted into body tissue by attaching the base body 2 to a skin of a user.

For detecting presence of an analyte in body tissue, the insertable analyte sensor 3 comprises a first electrode 6 and a second electrode 7, both, forming part of the insertable portion 4. The first electrode 6 as well as the second electrode 7 each comprises a first free end 6a, 7a and a second end 6b, 7b. The second end 6b of the first electrode 6 is electrically connected to first electric conductor 8. The electric conductor 8 electrically connects the first electrode 6 to the electronics unit 5. Likewise, the second end 7b of the second electrode 7 is electrically connected to a second electric conductor 9. The second electric conductor 9 electrically connects the second electrode 7 to the electronics unit 5, such that, during use of the medical device 1, an electric current can flow through the first electrode 6 and the second electrode 7.

The first electrode 6 extending from its second end 6b to the first free end 6a of the first electrode 6 as well as the second electrode 7 extending from its second end 7b to the first free end 7a of the second electrode 7 are configured to be, during use of the medical device 1, in electric contact with the body tissue of a user. The first electric conductor 8 and the second electric conductor 9 are not in electric contact with the body tissue, except of being electrically connected to the first electrode 6 and the second electrode 7, respectively.

For mitigating the risk of an uncontrolled flow of electric current in case of a short-circuit failure of the electronic units 5 or part thereof, the electrical resistance of the first electric conductor 8 and the electrical resistance of the second electric conductor 9 is higher than the electrical resistance of the first electrode 6 and the electrical resistance of the second electrode 7.

FIG. 2 shows another embodiment of a medical device 1 for detecting at least one analyte in a body fluid. This embodiment differs from the embodiment of the medical device 1 according to FIG. 1 in additionally comprising a power supply 11, here a battery. Battery 11 provides electric power to the electronics unit 5. For instance, battery 11 provides 3.4 V. The electronics unit 5 can be a printed circuit board.

The base body 2 further comprises patch 13, wherein patch 13 further comprises an adhesive for removably adhering the medical device 1 by patch 13 to a user's body.

Furthermore, the first electrode 6 and the second electrode 7 are carried by a carrier 12. Carrier 12 supports the first electrode 6 and the second electrode 7, and, thus, provides more stability to the first electrode 6 and the second electrode 7. Carrier 12 projects from the base body 2, and, during use of the medical device 1, is at least partially inserted into body tissue together with the first electrode 6 and the second electrode 7.

Base body 2 further comprises a bushing 10 for allowing the first electric conductor 8 and the second electric conductor 9 to reach from the electronics unit 5 through the base body 2 to the second end 6b of the first electrode 6 and to the second end 7b of the second electrode 7, respectively. The bushing 10 comprises a first side 10a and a second side 10b, wherein on the first side 10a of the bushing 10 there is the electronics unit 5 and wherein on the second side 10b, opposite to the first side 10a, of the bushing 10, there is the insertable portion 4 of the insertable analyte sensor 3.

The transition from the first electric conductor 8 to the first electrode 6 as well as the second electric conductor 9, i.e., the second end 6b of the first electrode 6 and the second end 7b of the second electrode 7, are located within the bushing 10. Thereby, bushing 10 protects the contact point between the first electric conductor 8 and the first electrode 6 as well as the contact point between the second electric conductor 9 and the second electrode 7.

A gain, the first electrode 6 extending from its second end 6b to its first free end 6a and the second electrode 7 extending from its second end 7b to its free end 7a are configured to be, during use of the medical device 1, in direct electric contact with the body tissue of a user. The electrical resistance of the first electrode 6 and the electrical resistance of the second electrode 7 each is 9 kOhm, wherein the electrical resistance of the first electric conductor 8 and the electrical resistance of the second electric conductor 9 is each 90 kOhm. Accordingly, the electrical resistance of the first electric conductor 8 and the second electric conductor 9 are equal to each other, and 10-times higher than the electrical resistance of the first electrode 8 and the second electrode 9. The electrical resistance of the first electrode 6 is equal to the electrical resistance of the second electrode 7.

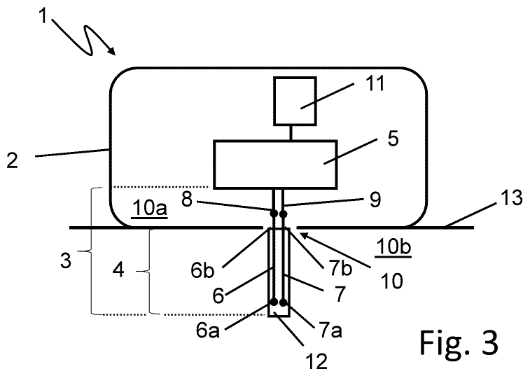

FIG. 3 shows a schematic view of another embodiment of a medical device 1 for detecting an analyte in a body fluid. The embodiment as shown in FIG. 3 differs from the embodiment of a medical device 1 as shown in FIG. 2 in that the second end 6b of the first electrode 6 and the second end 7b of the second electrode 7 are arranged on the first side 10a of the bushing 10.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

1 medical device
2 base body
3 insertable analyte sensor
4 insertable portion
5 electronics unit
6 first electrode
6a first free end of first electrode
6b second end of first electrode
7 second electrode
7a first free end of second electrode
7b second end of second electrode
8 first electric conductor
9 second electric conductor
10 bushing
10a first side of bushing
10b second side of bushing
11 power supply
12 carrier
13 patch

What is claimed is:

1. A medical device for detecting at least one analyte in a body fluid, comprising:
a base body configured for reversibly attaching the medical device to a patient's body;
an insertable analyte sensor comprising an insertable portion configured to be at least partially inserted into body tissue of a patient, the insertable analyte sensor comprising first and second electrodes both forming part of the insertable portion;
an electronics unit operably connected to the insertable analyte sensor; and
the first and second electrodes each comprising a first free end and a second end, the second end of the first electrode being electrically connected to a first electric conductor electrically connecting the first electrode to the electronics unit and the second end of the second electrode being electrically connected to a second electric conductor electrically connecting the second electrode to the electronics unit;
wherein the first electrode extending from the second end towards its first free end and the second electrode extending from the second end towards its first free end are configured to be, during use of the medical device, at least partially in electric contact with the body tissue of a patient;
wherein the electrical resistance of the first electric conductor and the electrical resistance of the second electric conductor is higher than the electrical resistance of the first electrode and the electrical resistance of the second electrode.

2. The medical device according to claim 1, wherein the sum of the electrical resistances of first electric conductor and the second electric conductor is higher than the sum of the electrical resistances of the first electrode and the second electrode.

3. The medical device according to claim 1, wherein the electrical resistance of the first electrode and the electrical resistance of the second electrode are equally high, and/or the electrical resistance of the first electric conductor and the electrical resistance of the second electric conductor are equally high.

4. The medical device according to claim 1, wherein the electrical resistance of the first electric conductor and the electrical resistance of the second electric conductor is one of at least 2-times, at least 4-times, at least 5-times and at least 10-times higher than the electrical resistance of the first electrode and the electrical resistance of the second electrode.

5. The medical device according to claim 1, wherein the first electric conductor comprises a first cable cross-sectional area, the second electric conductor comprises a second cable cross-sectional area, the first electrode comprises a third cable cross-sectional area, and the second electrode comprises a fourth cable cross-sectional area, wherein the first cable cross-sectional area and the second cable cross-sectional area are smaller than the third cable cross-sectional area and the fourth cable cross-sectional area.

6. The medical device according to claim 5, wherein the first cable cross-sectional area is smaller than the third cable cross-sectional area, and the second cable cross-sectional area is smaller than the fourth cable cross-sectional area.

7. The medical device according to claim 5, wherein the first cable cross-sectional area and the second cable cross-sectional area are of equal size, and/or the third cable cross-sectional area and the fourth cable cross-sectional area are of equal size.

8. The medical device according to claim 5, wherein the first cable cross-sectional area and the second cable cross-sectional area are one of at least 2-times, at least 4-times, at least 5-times, and at least 10-times smaller than the third cable cross-sectional area and the fourth cable cross-sectional area.

9. The medical device according to claim 1, wherein the base body comprises a bushing, wherein the electronics unit is arranged on a first side of the bushing and the insertable portion is arranged on a second side of the bushing opposite the first side, wherein the second end of the first electrode and the second end of the second electrode are both located within the bushing or at the first side of the bushing comprising the electronics unit or at the second side of the bushing comprising the insertable portion of the insertable analyte sensor.

10. The medical device according to claim 1, wherein the first electrode from its second end towards its first free end and the second electrode from its second end towards its first free end are, over their respective lengths, configured to be during use of the medical device in electric contact with the body tissue.

11. The medical device according to claim 1, wherein the first electric conductor and the second electric conductor are, over their respective lengths, configured to be during use of the medical device electrically isolated against the body tissue.

12. The medical device according to claim 1, wherein the medical device comprises a power supply configured for providing electric power to the electronics unit.

13. The medical device according to claim 12, wherein the electrical resistance of the first electric conductor and the electrical resistance of the second electric conductor are selected so that the voltage of the power supply divided by the sum of the electrical resistance of the first electrical conductor and the electrical resistance of the second electric conductor is smaller than or equal to 50 μA.

14. The medical device according to claim 1, wherein the insertable portion comprises a carrier configured for carrying the first electrode and the second electrode.

15. The medical device according to claim 1, wherein the base body comprises a patch that has an adhesive configured for adhering the medical device to a patient's body.
